(19) United States
(12) Patent Application Publication
(10) Pub. No.: US 2013/0302445 A1
(43) Pub. Date: Nov. 14, 2013

(54) APPARATUS AND METHOD FOR TREATING CEREBRAL ISCHEMIA USING NON-INHALED CARBON DIOXIDE

(71) Applicants: Denise Barbut, New York, NY (US); Allan Rozenberg, San Diego, CA (US); Axel Heinemann, New York, NY (US)

(72) Inventors: Denise Barbut, New York, NY (US); Allan Rozenberg, San Diego, CA (US); Axel Heinemann, New York, NY (US)

(21) Appl. No.: 13/863,919

(22) Filed: Apr. 16, 2013

Related U.S. Application Data
(60) Provisional application No. 61/624,484, filed on Apr. 16, 2012.
(60) Provisional application No. 61/661,709, filed on Jun. 19, 2012.
(60) Provisional application No. 61/720,164, filed on Oct. 30, 2012.

(51) Int. Cl. A61K 9/00

(52) U.S. Cl. CPC A61K 9/0043
USPC 424/700; 514/509; 604/23

(57) ABSTRACT

A non-invasive method of treating cerebral ischemia, involving the use of non-inhaled, intra-nasally delivered carbon dioxide (CO2), alone or in combination with other gases to augment cerebral perfusion and improve outcome following a stroke is provided. A vasodilator gas is delivered intranasally, alone or in combination with a second gas, for prolonged periods of time without systemic absorption. The second gas may be selected from NO, hydrogen, xenon, anesthetic gases, oxygen, nitrogen, nitrous oxide, carbon monoxide, or air. The treatment selectively increases cerebral perfusion and provides neuroprotection in the treatment of cerebral ischemia.

### CROSS REFERENCE TO RELATED APPLICATIONS

This application claims the benefit of priority to U.S. Appln. Ser. No. 61/624,484 filed on Apr. 16, 2012 and U.S. Appln. Ser. No. 61/661,709 filed on Jun. 19, 2012 and U.S. Appln. Ser. No. 61/720,164 filed on Oct. 30, 2012, the entireties of which are hereby incorporated by reference.

### FIELD OF THE INVENTION

The present invention related to a non-invasive apparatus and method of threating cerebral ischemia or trauma. In particular, the invention involves the use of non-inhaled, intra-nasally or intra-orally delivered CO2 to augment cerebral perfusion and improved outcome following stroke.

### BACKGROUND OF THE INVENTION

Stroke occurs when focal cerebral ischemia is severe, prolonged or both. Cerebral perfusion augmentation early in the ischemic event improves outcome from stroke. One method to accomplish this is to elevate blood pressure but this carries the risk of cerebral hemorrhage. Furthermore, pharmacologic hypertension triggers autoregulatory responses and may actually reduce cerebral perfusion.

Pharmacologic manipulation of cerebral vascular tone has also been attempted using systemic vasodilators such as nitroprusside and nitroglycerin. Nitroprusside is a potent vasodilator but marked systemic hypotension overrides any beneficial effect it might have on the cerebral circulation. It has even been given concomitantly with vasoconstrictors such as epinephrine, but the latter leads to constriction of the cerebral vasculature. These complicated pharmacodynamics have prevented systemic hypotensive agents being used in the treatment of cerebral ischemia.

Several devices have recently been used to augment perfusion to the ischemic tissue. Clot retraction or thrombectomy, using a variety of devices, is used to remove the clot occluding the artery but the technique is associated with higher hemorrhage rates in the core, especially in patients treated beyond the first few hours of ischemia. Furthermore, vessel occlusion is not present angiographically in many patients. For all these reasons, thrombectomy is performed in fewer than 1% of strokes.

Partial aortic occlusion has also been shown to increase cerebral perfusion during ischemia when performed early and is not associated with increased cerebral hemorrhage rates. It is however, an invasive procedure requiring trained personnel. Vagal nerve stimulation may improve outcome from stroke in animals but has not been shown to do so in humans. The mechanism of action has not been elucidated though it may involve a dampening of the inflammatory cascade rather than an increase in cerebral blood flow (cbf). Electrical stimulation of the sphenopalatine ganglion, the ganglion for intracranial parasympathetic fibres, may also increase cbf but its benefit in stroke has not yet been determined and furthermore, like all other existing stroke treatments, it is an invasive procedure performed by interventional neuroradiologists.

None of the foregoing methods have been effective in improving the outcome from stroke. Further, the foregoing methods attempting to improve the outcome following stroke are invasive procedures requiring trained personnel. Thus, improvements over conventional therapies are desirable.

### BRIEF SUMMARY OF THE INVENTION

The present invention relates to a new non-invasive method of treating cerebral ischemia, involving the use of non-inhaled, intra-nasally delivered carbon dioxide (CO2), alone or in combination with other gases to augment cerebral perfusion and improve outcome following a stroke.

Intranasal delivery of CO2 focally for prolonged periods of time and without systemic absorption for improving outcome from cerebral ischemia has not been previously described. This procedure is completely non-invasive and avoids complications of gas inhalation.

The vasodilator gas alone or in combination with a second gas is delivered intranasally for prolonged periods of time without systemic absorption. The second gas is selected from the group consisting of NO, hydrogen, xenon, anesthetic gases, oxygen, nitrogen, nitrous oxide, carbon monoxide, or air. The treatment selectively increases cerebral perfusion and provides neuroprotection in the treatment of cerebral ischemia.

### BRIEF DESCRIPTION OF THE DRAWINGS

For a better understanding of the invention, and to show how the same may be carried into effect, reference will now be made, by way of example, to the accompanying drawings, in which:

FIG. 1 is an illustration of the nasal cavity depicting the delivery of intranasal gas with separate gas delivery into the trachea in accordance with the invention.

FIG. 2 is a graph illustrating that cbf is more than doubled during intranasal or intra-oral delivery of CO2.

FIG. 3 is a graph illustrating that the increase in cbf is attributable to focal intranasal or intra-oral effect rather than to systemic absorption of CO2.

FIG. 4 depicts the human tolerability of intranasal administration of CO2 at various concentrations.

FIG. 5 is an illustration depicting anterior nasal occlusion to prevent inhalation of CO2 in accordance with an exemplary embodiment of the invention.

FIG. 6 is an illustration depicting the anterior nasal occlusion of FIG. 2 combined with a one-way valve or suction catheter.

FIG. 7 is a view of an exemplary device in accordance with the invention.

FIG. 8 illustrates the exemplary device of FIG. 7 with posterior nasopharyngeal occlusion showing trans-nasal diffusion of the gas into the brain.

FIG. 9 is an illustration depicting the setup for the method of delivery in accordance with the invention.

FIG. 10 is a graph showing the effect of intranasal, non-inhaled CO2 on cerebral blood flow in rats with posterior nasopharyngeal occlusion.

FIG. 11 is a graph depicting CO2 systemic challenge and intranasal dose response.

FIG. 12 is a graph depicting the perfusion recovery in a rat during cerebral ischemia without non-inhaled NO as compared to the perfusion recovery of a rat receiving non-inhaled NO during cerebral ischemia.

### DETAILED DESCRIPTION OF THE INVENTION

As used herein, cerebral ischemia should be understood in its broadest sense and incorporate both focal and global cerebral ischemia.

Intranasal delivery of CO2 focally for prolonged periods of time and without systemic absorption for improving outcome from cerebral ischemia has not been previously described. This procedure is completely non-invasive and avoids complications of gas inhalation.

The vasodilator gas alone or in combination with a second gas is delivered intranasally for prolonged periods of time without systemic absorption. The second gas is selected from the group consisting of hydrogen, xenon, anesthetic gases, oxygen, carbon monoxide, or air. The treatment selectively increases cerebral perfusion and provides neuroprotection in the treatment of cerebral ischemia.

Puffs of CO2 have been given intranasally to ameliorate allergic rhinitis and migraine symptoms, possibly through trigeminal stimulation (U.S. Pat. No. 7,748379). These patients must cooperate and be trained to not inhale through their nose during treatments which only lasts a few seconds. In such tiny doses lasting only seconds, only very small amounts of CO2 will in fact be inhaled and systemic effects of this inhalation will be negligible. However, if the CO2 treatment period exceeds this short time, and continues for 30-60 minutes as envisaged in this filing, then inhalation of CO2 will be manifest and systemic arterial gas and pH changes be evident, as will the physiological consequences of CO2 inhalation. What we are proposing to do is different. We will be delivering the vasodilator gas alone or in combination for prolonged periods of time without any systemic absorption and for selectively increasing perfusion in the treatment of cerebral ischemia.

Systemically delivered CO2 gas is a very potent cerebral vasodilator. In intubated patients, when CO2 is allowed to rise by reducing the ventilation rate, cbf rises rapidly and linearly in response to hypercarbia, such that every mm Hg increase in arterial CO2 over 45 mm is associated with a 4% increase in cbf. CO2 is very soluble and diffuses readily through the blood brain barrier (BBB) and from the intravascular space intracranially to the cerebrospinal fluid (csf). The mechanism of action is through formation of hydrogen ions which then stimulate nitric oxide formation in cerebral vessels. Hydrogen ions also stimulate the chemoreceptor trigger zone (CTZ) in the anterior medulla which leads to neurogenic vasodilation.

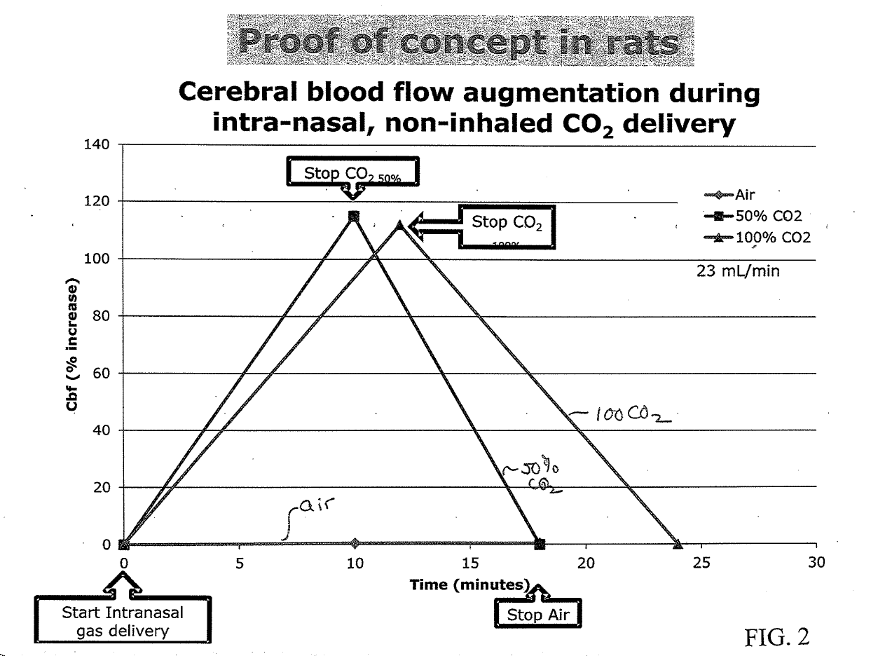

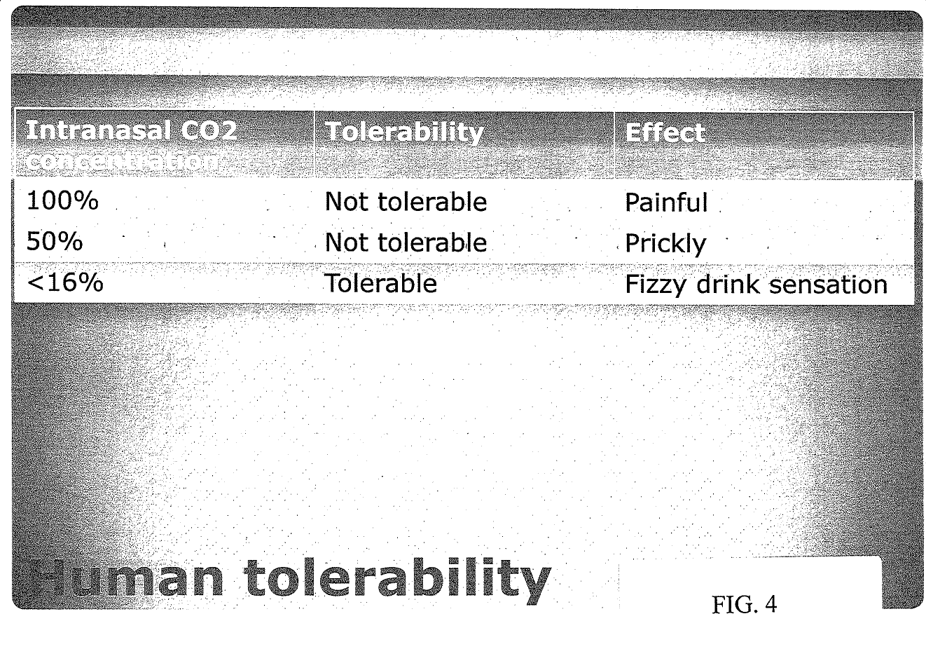

When CO2 s inhaled through the nose or mouth into the pulmonary circulation, it passes into the systemic blood stream through the alveoli. Once it reaches the brain, it diffuses out into the csf to exert its effect. As more CO2 is inhaled into the lungs and absorbed into the systemic circulation, arterial CO2 concentration (PaCO2) rises, hydrogen ions are produced and arterial pH falls, creating an acidosis. Acidosis leads to narcosis, and ultimately coma and death. Less severe signs of CO2 inhalation in non-intubated patients include headaches, sweating and hyperventilation. We have demonstrated that CO2 augments cbf when delivered intranasally but without inhalation into the lungs in the rat as illustrated in FIGS. 2-4. Inhalation is prevented by ventilating the animal through a tracheostomy with air while delivering the experimental gas intranasally (or intra orally).

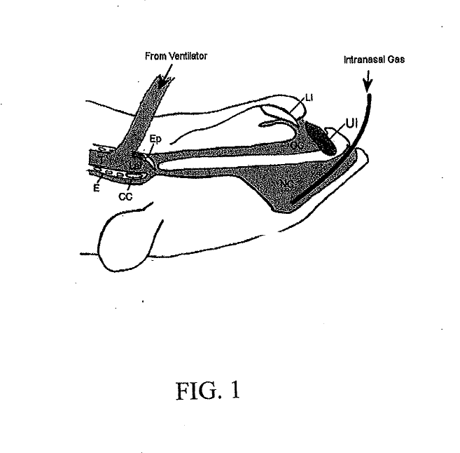

Referring now to FIG. 1 an illustration of the nasal cavity depicting the delivery of intranasal gas in accordance with the invention.

As can be seen in FIG. 2 cbf has more than doubled during intranasal or intra-oral delivery of CO2. The increase is the same with 100% and with 50% gas concentration. The increase begins within seconds of delivery and continues as long as delivery is continued, returning to baseline following discontinuation of delivery. Decline following discontinuation is somewhat slower than rise during delivery. When air is delivered intranasally or intra-orally at similar flow rates, cbf does not increase indicating that the increase in cbf was not caused by the gas flow alone but that it was specific to CO2.

The volumes of CO2 delivered intra nasally and without inhalation to achieve an increase in cbf are very small compared to those required to produce the same amount of cbf increase by inhalation into the systemic circulation. Because of this, the acidosis and ultimate stupor , coma and death which would occur following CO2 inhalation would not occur if CO2 were delivered focally into the nose, close to the cerebral circulation and without the need for inhalation.

Assuming a minute volume of 6 liters per minute and a 50 mL per minute flow of 16% CO₂ into the nasal cavity, if all the CO₂ is inhaled, the effective increase in alveolar CO2 would be 0.13% or 1 mmHg which would have practically no effect on arterial pH.

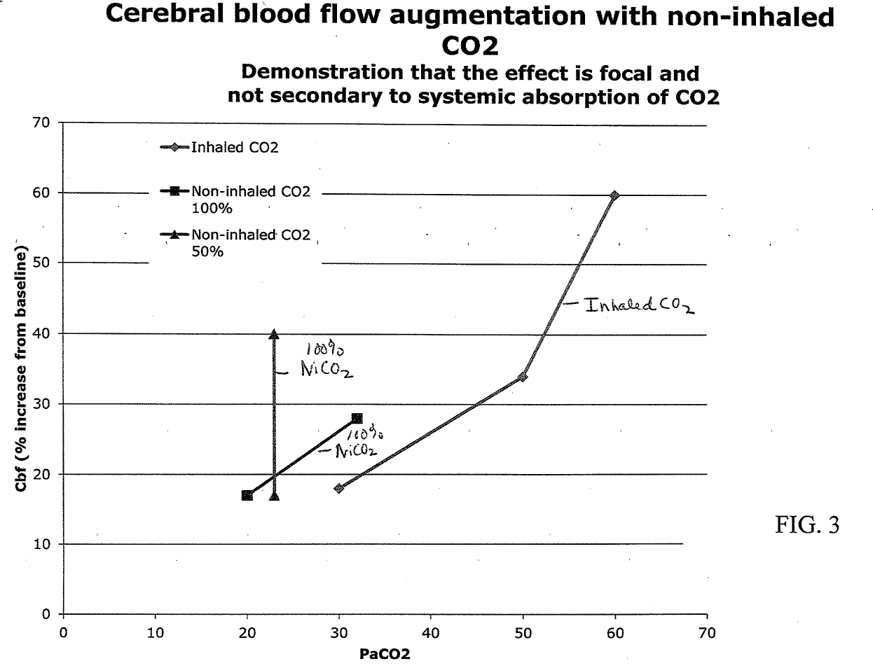

Referring now to FIG. 3, it can be seen that the increase in cbf is attributable to focal intranasal or intra-oral effect rather than to systemic absorption of CO2. When CO2 is delivered into the lungs, cbf increases sharply, in an exponential fashion. During such delivery, arterial CO2 concentration rapidly increases and arterial pH falls. When 100% CO2 is delivered intranasally, cbf also increases but with far less increase in systemic CO2. At this very high concentration, sufficient CO2 must be absorbed focally, intracranially, into the cerebral circulation to appear in measurable amounts in the systemic circulation. It cannot be concluded, therefore, that the increase in cbf is caused by a purely local action of CO2 rather than being secondary to systemic absorption. However, when intranasal CO2 concentration is reduced to 50%, cbf doubles without any detectable increase in systemic PaCO2 or fall of pH. The effect on cbf during intranasal or intra oral delivery of CO2 with concomitant air inhalation into the lungs is therefore a focal effect on intranasal and intracranial vasculature rather than secondary to systemic absorption. This has not been previously demonstrated and is fundamental to the discovery set forth in this patent.

While CO2 may be given to intubated, comatose individuals, the tolerability of intranasal gas delivery in awake humans has not previously been investigated. We have systematically tested the tolerability and effect of CO2 during intranasal delivery. Results are shown in FIG. 3. CO2 elicits intense burning and stinging sensations which are intolerable above 16%. At that concentration or below, the mild stinging or tingling sensations elicited are quite tolerable and abate over a few minutes. So individuals who are awake, such as stroke patients, these lower concentrations would be recommended. The current invention delivers CO2, or a combination of gases into the nasal or oral cavity but prevents its inhalation for the purposes of improving outcome from cerebral ischemia. The passage of the gases into the lungs could be prevented by placing a nasal “clip” or otherwise occluding the entrance to the nostrils, proximal to where the gas is delivered or by one or two posterior inflatable plugs occluding the choana (outlet from nasal cavity to pharynx) or delivering small enough quantities which may be significantly diluted by respiratory gases to not have much effect on the systemic circulation. In the embodiment with the anterior occlusion or “clip,” the CO2 could escape via a valve or exit tube or be suctioned out into a vacuumed container.

In yet another embodiment, CO2 could be delivered with another vasodilator drug in topical ointment form, such as glyceryl trinitrate. The ointment and the CO2 gas could enhance each other's absorption across the nasal mucosa.

CO2 may also affect the outcome from cerebral ischemia or trauma by mechanisms other than cbf augmentation. More specifically, it might have a neuroprotective action of its own when delivered intranasally, rather than by inhalation. The mechanism of action might be inhibition of glutamate/NMDA-mediated excitotoxicity or by caspase/interleukin/TNFalpha-mediated inflammation later in the course of the insult. Neuroprotection with systemic hypercarbia (following CO2 inhalation or hypoventilation) may be available even when inhaled for only 2 hours.

In yet another embodiment, CO2 could be delivered with another neuroprotective gas such as carbon monoxide. Carbon monoxide (CO) may also be neuroprotective following ischemic or traumatic injury.

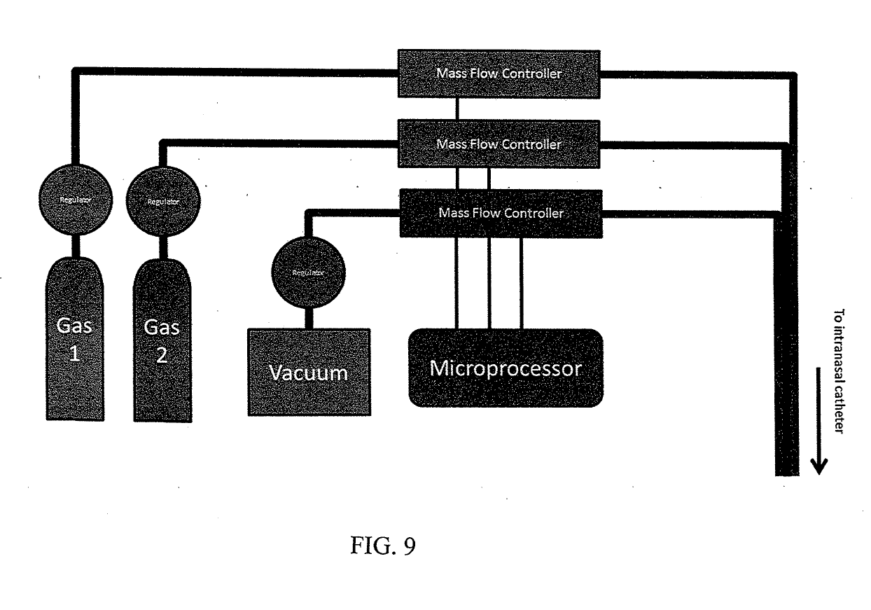

As best seen in FIG. 9 The equipment for the delivery of CO2 to stroke patients may include a small cartridge of CO2, a small cartridge of air (or other gas), a disposable multi-lumen catheter to deliver the gases into the nasal cavity where the mixing would occur inside or outside the nose, the clip (or posterior balloons over-the-wire), the valve or tube to let the gas out with suction if necessary, the vacuum container for suctioned secretions, and a flow regulator to determine flow rate and maybe adjust CO2 concentration. The entire apparatus may also be modified such that CO2 is only delivered through one nostril because only small volumes are required to increase cbf and cerebral vasodilation. In that gas, only one nostril would need to be occluded to prevent inhalation, while the patient would be free to breathe in the other nostril. In yet another embodiment, the gas could be delivered through a face mask. In that case, assuming inhalation is not prevented by intubation, posterior balloon occlusion would be required.

The combination of CO2 and of other gases might have a synergistic effect on cbf. Furthermore, this would permit further reduction in the concentrations or volumes required to achieve the same effect.

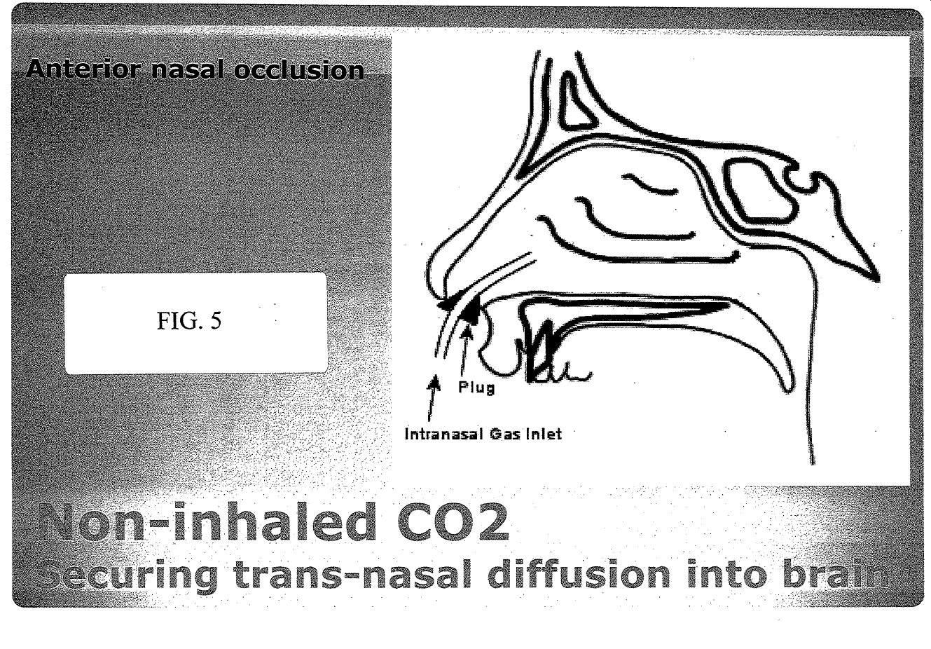

Referring to FIG. 5, an illustration depicting anterior nasal occlusion to prevent inhalation of CO2 in accordance with an exemplary embodiment of the invention is shown. The anterior occlusion may be an inflatable balloon, a pre-configured plug, a nasal clip or other like elements known to those of skill in the art. Gas delivery is achieved through the nasal catheter crossing the plug. In another exemplary embodiment, delivered gas may be suctioned out through a separate tube in the same or opposite nostril to the delivery of gas. In yet another exemplary embodiment, a one way valve may prevent inhalation but allow gas to be blown out of the nostrils.

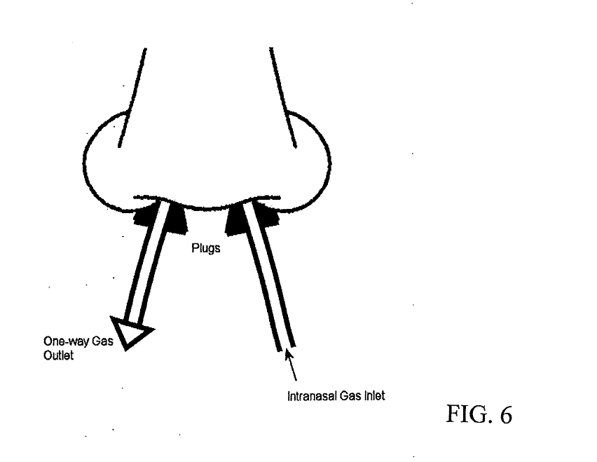

FIG. 6 is an illustration depicting the anterior nasal occlusion of FIG. 2 combined with a one-way valve or suction catheter.

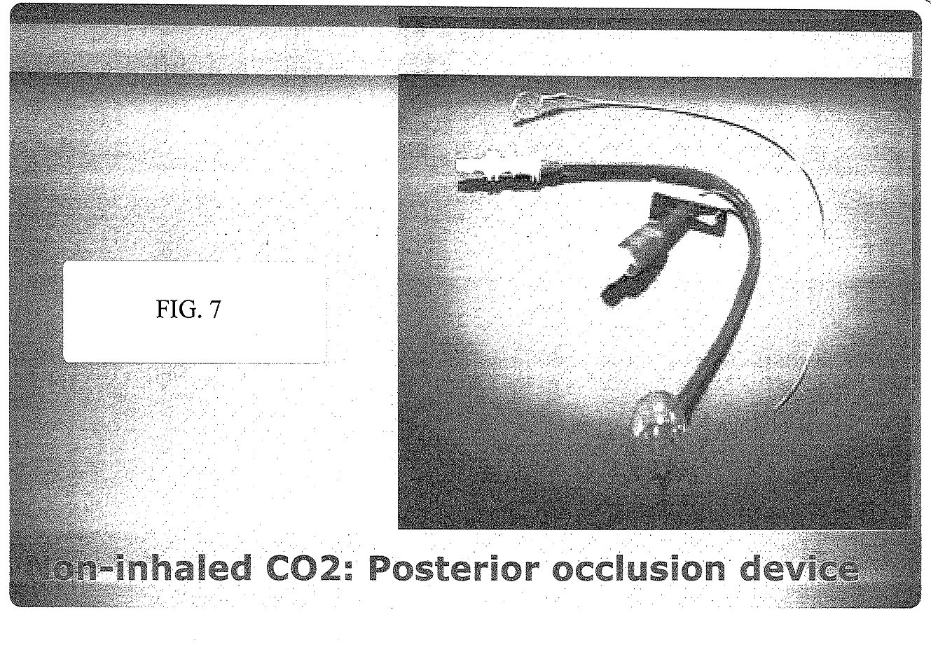

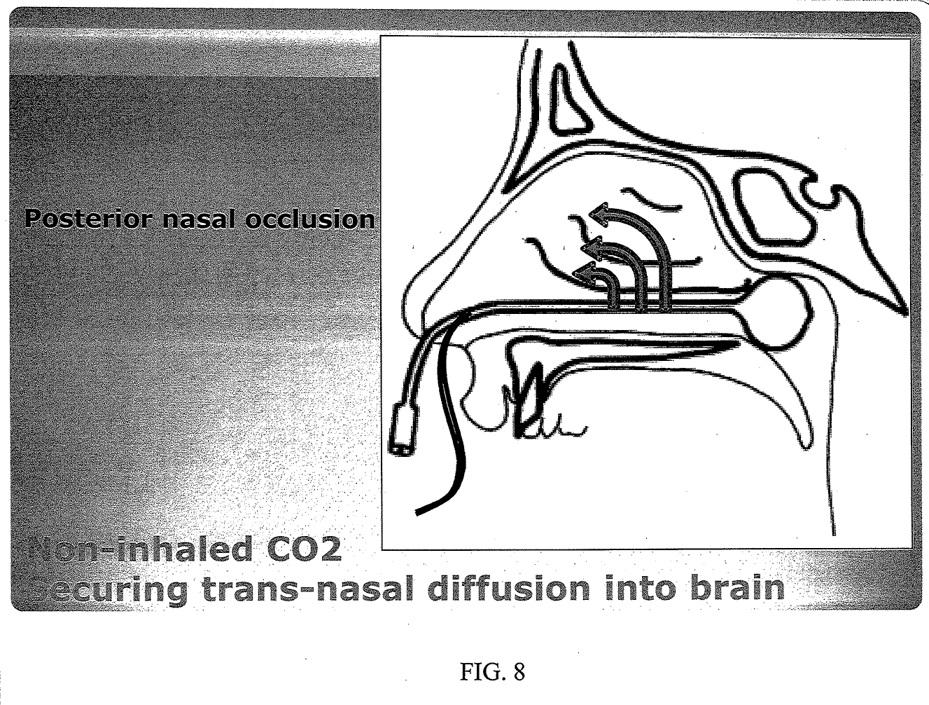

FIG. 7 is a view of an exemplary device in accordance with the invention while FIG. 8 illustrates the exemplary device of FIG. 7 with posterior nasopharyngeal occlusion. This embodiment shows the posterior occlusion device. It includes a guide wire over which the gas delivery catheter is inserted, the lumen for balloon inflation, several other lumina for CO2 delivery as well as delivery of other gases, and the inflatable balloon. The gases can be allowed to mix prior to delivery or at the point of delivery.

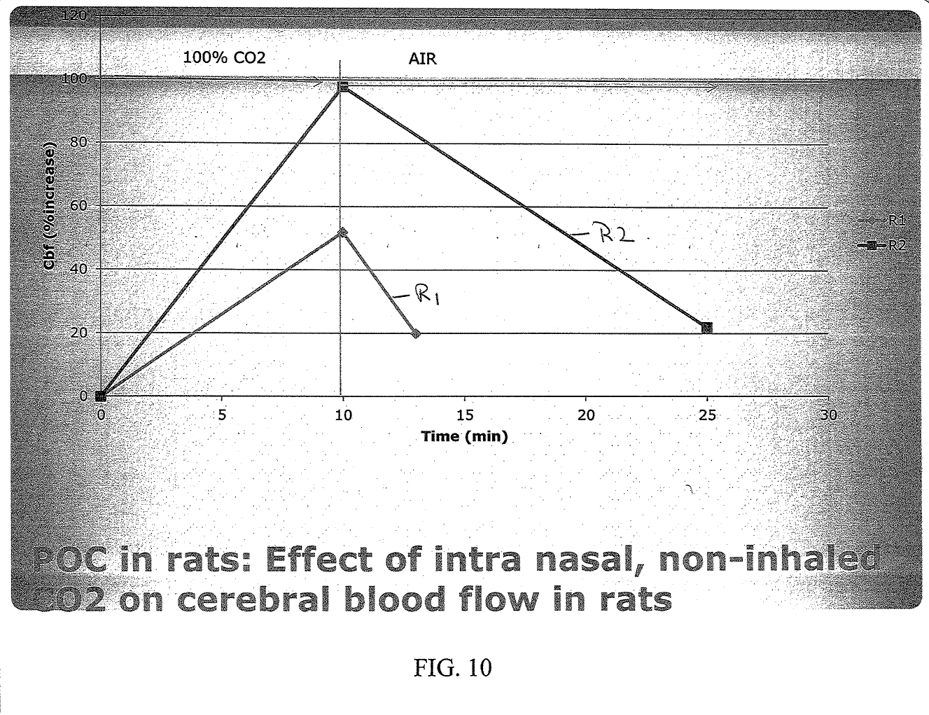

FIG. 9 shows an exemplary set up for delivery of non-inhaled gas in accordance with the invention. FIG. 10 is a graph showing the effect of intranasal, non-inhaled CO2 on cerebral blood flow in rats with posterior nasopharyngeal occlusion.

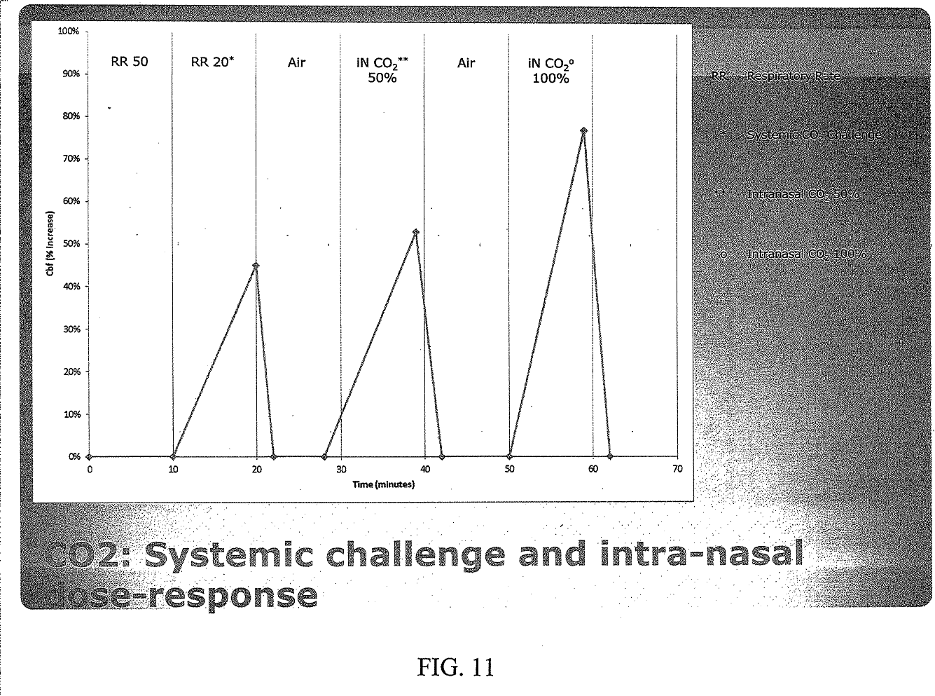

FIG. 11 is a graph depicting CO2 systemic challenge and intranasal dose response.

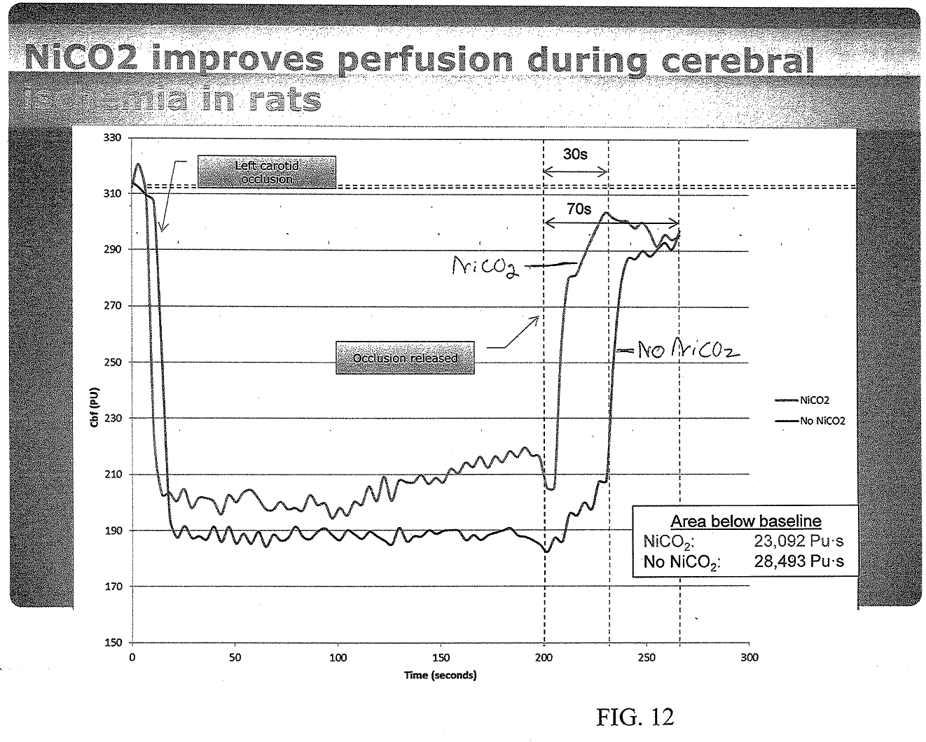

FIG. 12 is a graph depicting the perfusion recovery in a rat during cerebral ischemia without non-inhaled CO2 as compared to the perfusion recovery of a rat receiving non-inhaled CO2 during cerebral ischemia.

Volatile anesthetics, arginine and other gases such as hydrogen, and other gases known to those skilled in the art, may also stimulate the trigeminal nerve and the autonomic fibres travelling with it, when delivered as gases into the nasal or oral cavity without being inhaled. Any one of these could be given alone or in combination with CO2 to improve outcome from cerebral ischemia.

Lastly, non-inhaled CO2, alone or in combination, might be useful for the treatment of transient ischemic attacks (TIA) and vasospasm. In the case of TIA, a separate embodiment would be used outside a hospital setting. In this embodiment, the CO2 would be in an inhaler, and the gas release would only occur once the nasal plugs were properly positioned. This embodiment could also be used to increase cerebral blood flow transiently, when attention or memory needed to be improved. Cerebral blood flow diminishes with age and the elderly could benefit from periodic boosts.

Various modifications and additions may be made to the exemplary embodiments disclosed herein without departing from the scope of the invention. For example, while the embodiments disclosed herein refer to particular features, the scope of this invention also includes embodiments having different combinations of features and embodiments that do not include all of the described features. Accordingly, the scope of the present invention is intended to embrace all such alternative, modifications and variations as fall within the scope of the claims and equivalents thereof.

What is claimed is:

1. A method for improving outcome following cerebral ischemia in a patient comprising:
inserting an elongate tubular member into the nasal cavity, the elongate tubular member having a proximal end, and a distal end, a lumen extending there between, the lumen communicating with a port on a distal region of the elongate tubular member;
plugging the nares of the patient to block inhalation through the nose; and
delivering a gas into the nasal cavity for direct absorption into the brain through the nasal vasculature while preventing the inhalation of said gas so as to not significantly alter the systemic arterial levels of the gas, its metabolites or pH.

2. The method of claim 1, wherein the gas is CO2.

3. The method of claim 1, wherein a flow rate of gas is between 1-200 mL/min.

4. The method of claim 1, wherein a time of use is between 1 minute and 96 hours.

5. The method of claim 1, wherein delivering a gas into the nasal cavity includes occluding the nasal cavity to prevent inhalation through the nose.

6. The method of claim 1, wherein delivering a gas into the nasal cavity includes delivering the gas through an elongate tubular member.

7. The method of claim 1, wherein delivering a gas into the nasal cavity includes delivering the gas via a facemask.

8. The method of claim 1, wherein plugging the nares of the patient includes occluding the nares proximal to a gas delivery port.

9. The method of claim 1, wherein plugging the nares of the patient includes occluding the nasal cavity distal to a gas delivery port.

10. The method of claim 5, wherein occluding the nasal cavity includes providing an inflatable expandable member for occluding the nasal cavity.

11. The method of claim 50, wherein occluding the nasal cavity includes providing a rigid member for occluding the nasal cavity.

12. The method of claim 2, wherein the concentration of CO2 is between 5 and 100%.

13. The method of claim 2, wherein the concentration of CO2 is between 1 and 200 ppm.

14. The method of claim 6, wherein a neurostimulating electrode is attached to the tubular member.

15. The method of claim 1, wherein delivering the gas is continuous.

16. The method of claim 1, wherein delivering the gas is intermittent.

17. The method of claim 1, wherein delivering the gas includes delivering multiple gases simultaneously.

18. The method of claim 6, wherein the elongate tubular member has multiple lumens to deliver multiple gases.

19. The method of claim 17, wherein the gas contains hydrogen or xenon.

20. The method of claim 17, wherein the gas contains oxygen.

21. The method of claim 17, wherein the gas contains a volatile anesthetic.

22. The method of claim 17, wherein the flow of each gas can be individually controlled.

23. The method of claim 1, wherein the gas flow can be controlled by feedback from a cerebral blood flow monitor.

24. The method of claim 1, wherein the gas flow can be controlled by feedback from a blood pH monitor.

25. The method of claim 1, wherein the gas flow can be controlled by feedback from a patient carbon dioxide monitor.

26. The method of claim 1, wherein the gas delivered is at room temperature.

27. The method of claim 1, wherein the gas delivered is above room temperature.

28. The method of claim 1, wherein the gas is humified prior to or during delivery.

29. A method for cerebral blood flow augmentation during cerebral ischemia by delivery of a gas into the nasal or oral cavity while minimizing the inhalation of said gas so as to not significantly alter the systemic arterial carbon dioxide or pH levels.

30. The method of claim 29, wherein the gas is carbon dioxide.

31. The method of claim 29, wherein the flow rate of gas is between 1-200 mL/min.

32. The method of claim 29, wherein the time of use is between 1 minute and 96 hours.

33. The method of claim 29, wherein the nasal cavity is occluded to prevent inhalation through the nose.

34. The method of claim 29, wherein the gas is delivered through an elongate tubular member.

35. The method of claim 29, wherein the gas is delivered via a facemask.

36. The method of claim 29, wherein the nares are occluded proximal to the gas delivery port.

37. The method of claim 29, wherein the nasal cavity is occluded distal to the gas delivery port.

38. The method of claim 37, wherein the occlusion is an inflatable expandable member.

39. The method of claim 37, wherein the occlusion is a rigid member.

40. The method of claim 30, wherein the concentration of carbon dioxide is between 5 and 100%.

41. The method of claim 30, wherein the concentration of carbon dixoide is between 1 and 200 ppm.

42. The method of claim 34, wherein a neurostimulating electrode is attached to the tubular member.

43. The method of claim 29, wherein the gas is delivered continuously.

44. The method of claim 29, wherein the gas is delivered intermittently.

45. The method of claim 29, wherein multiple gases are delivered simultaneously.

46. The method of claim 34, wherein the elongate tubular member has multiple lumens to deliver multiple gases.

47. The method of claim 29, wherein the gas contains hydrogen or xenon.

48. The method of claim 29, wherein the gas contains oxygen.

49. The method of claim 29, wherein the gas contains a volatile anesthetic.

50. The method of claim 45, wherein the flow of each gas can be individually controlled.

51. The method of claim 29, wherein the gas flow can be controlled by feedback from a cerebral blood flow monitor.

52. The method of claim 29, wherein the gas flow can be controlled by feedback from a blood pH monitor.

53. The method of claim 29, wherein the gas flow can be controlled by feedback from a patient carbon dioxide monitor.

54. The method of claim 29, wherein the delivery of gas includes gas delivered at room temperature.

55. The method of claim 29, wherein the delivery of gas includes gas delivered above room temperature.

56. The method of claim 29, wherein the delivery of gas includes gas that is humified prior to or during said delivery.

57. A method of suppressing the trigeminal cholinergic inflammatory cascade for the treatment of inflammatory diseases such as rheumatoid arthritis, inflammatory bowel disease, neurogenic inflammatory conditions such as acute respiratory distress syndrome (ARDS) includes delivering a gas into the nasal or oral cavity while minimizing the inhalation of said gas so as to not significantly alter the systemic arterial carbon dioxide or pH levels.

58. The method of claim 57, wherein the gas is CO2.

59. The method of claim 57, wherein the flow rate of gas is between 1-200 mL/min.

60. The method of claim 57, wherein the time of use is between 1 minute and 96 hours.

61. The method of claim 57, wherein the nasal cavity is occluded to prevent inhalation through the nose.

62. The method of claim 57, wherein delivering a gas is through an elongate tubular member.

63. The method of claim 57, wherein delivering a gas is via a facemask.

64. The method of claim 57, wherein minimizing the inhalation of gas includes occluding the nares proximal to a gas delivery port.

65. The method of claim 57, wherein minimizing the inhalation of gas includes occluding the nasal cavity distal to a gas delivery port.

66. The method of claim 57, wherein minimizing the inhalation of gas includes occluding the nasal or oral cavity with an inflatable expandable member.

67. The method of claim 57, wherein minimizing the inhalation of gas includes occluding the nasal or oral cavity with a rigid member.

68. The method of claim 58, wherein the concentration of carbon dioxide is between 5 and 100%.

69. The method of claim 58, wherein the concentration of carbon dioxide is between 1 and 200 ppm.

70. The method of claim 62, wherein a neurostimulating electrode is attached to the tubular member

71. The method of claim 57, wherein delivering the gas is continuous.

72. The method of claim 57, wherein delivering the gas intermittent.

73. The method of claim 57, wherein delivering the gas includes delivering multiple gases simultaneously.

74. The method of claim 62, wherein the elongate tubular member includes multiple lumens to deliver multiple gases.

75. The method of claim 73, wherein the gas contains hydrogen or xenon.

76. The method of claim 73, wherein the gas contains oxygen.

77. The method of claim 73, wherein the gas contains a volatile anesthetic.

78. The method of claim 73, wherein the flow of each gas can be individually controlled.

79. The method of claim 57, wherein the gas flow can be controlled by feedback from a cerebral blood flow monitor.

80. The method of claim 57, wherein delivering the gas can be controlled by feedback from a blood pH monitor.

81. The method of claim 57, wherein delivering the gas can be controlled by feedback from a patient carbon dioxide monitor.

82. The method of claim 57, wherein delivering the gas includes delivering the gas at room temperature.

83. The method of claim 57, wherein delivering the gas includes delivering the gas above room temperature.

84. The method of claim 57, wherein delivering the gas includes humidifying the gas prior to or during delivery.

85. A medical device for increasing cerebral blood flow comprising a nasal gas catheter; a gas and a means to control the flow of such gases.

86. The device of claim 85, wherein the gas is carbon dioxide.

87. The device of claim 85, wherein the gas is a vasodilator such as anesthetic gases.

88. The device of claim 85, wherein the gas is a mixture of gases.

89. The device of claim 88, wherein the gas contains nitrogen.

90. The device of claim 88, wherein the gas contains oxygen.

91. The device of claim 88, wherein the gas contains a volatile anesthetic, xenon, hydrogen, nitrous oxide.

92. The device of claim 85, wherein the device is configured to occlude the nasal cavity to prevent inhalation through the nose.

93. The device of claim 85 further comprising an elongate tubular member for delivering the gas to the nasal cavity.

94. The device of claim 85 further comprising a facemask for delivering the gas to the nasal cavity.

95. The device of claim 92, wherein the device is configured to occlude the nares proximal to a gas delivery port.

96. The device of claim 92, wherein the device is configured to occlude the nasal cavity distal to a gas delivery port.

97. The device of claim 92, wherein the occlusion is an inflatable expandable member.

98. The device of claim 92, wherein the occlusion is a rigid member.

99. The device of claim 93, wherein the gas is delivered through the end of the tubular member.

100. The device of claim 93, wherein the gas is delivered through one or more ports along a longitudinal axis of the tubular member.

101. The device of claim 93, wherein the tubular member includes a plurality of lumens each configured to deliver a different gas.

102. The device of claim 93, wherein the tubular member includes a plurality of lumens each configured to carry a separate gas, said plurality of lumens configured to converge into one lumen prior to delivery of said separate gases.

103. The device of claim 102, wherein one or more lumens have suction applied to them to prevent inhalation by having the net outflow from the nasal cavity be higher than the sum of the inflow into the nasal cavity.

104. The device of claim 85, wherein the flow of different gases are controlled by valves.

105. The device of claim 85, wherein the flow of the gas is controlled based on patient feedback.

106. The device of claim 105, wherein the feedback is cerebral blood flow.

107. The device of claim 105, wherein the feedback is arterial pH.

108. The device of claim 105, wherein the feedback is arterial carbon dioxide.

109. The device of claim 105, wherein the feedback is end tidal carbon dioxide.

110. The device of claim 85, wherein the gas is delivered by an inhaler with anterior occluding nasal plugs.

111. A method of delivering carbon dioxide by coating the nasal mucosa with glyceryl trinitrate.

112. A method of increasing cerebral blood flow in people with attention or memory disorder by delivering a gas into the nasal cavity for direct absorption into the brain through the nasal vasculature while preventing the inhalation of said gas so as to not significantly alter the systemic arterial levels of the gas, its metabolites or pH.
